(19) United States
(12) Patent Application Publication
(10) Pub. No.: US 2005/0228282 A1
(43) Pub. Date: Oct. 13, 2005

(54) Image quality compensation for duplex or triplex mode ultrasound systems

(75) Inventors: Hong Wang, Bellevue, WA (US); John R. Klepper, Seattle, WA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/820,106

(22) Filed: Apr. 6, 2004

(51) Int. Cl. A61B 8/02

(52) U.S. Cl. 600/453

(57) ABSTRACT

Duplex operation for ultrasound imaging uses a single fixed voltage level. Both B-mode and color-mode imaging are performed using a power level that may be less than the power level for single-mode B-mode imaging, and may be greater than the power level for single-mode color-mode imaging. A duty cycle of color-mode pulses may be reduced. To enhance image quality, the number of cycles in the B-mode pulse profile may be increased. To enhance image quality, the center frequency of the B-mode pulse profile may be reduced, and dynamic filtering for B-mode received pulses may be appropriately modified.

### BACKGROUND

The present invention relates generally to ultrasound imaging and specifically to methods for ultrasound transmission that enable multiple modes of imaging. Generally, different imaging modes require different voltage levels for transmission of appropriate instantaneous power levels. Different voltage levels require a multiplicity of voltage supplies, which adds to system complexity.

Ultrasound imaging systems generate a sequence of images representing a region of a body. Ultrasound imaging has been widely used to observe tissue structures within a human body, such as the heart structures, the abdominal organs, the fetus, and the vascular system. In general, an ultrasound imaging system includes transducers connected to beamformers for the transmission and reception of signals. A transmit beamformer applies electrical pulses to a transducer in a predetermined timing sequence to generate transmit beams that propagate in predetermined directions from the transducer into the body. The transmit beams are generally created with frequencies in the range of 2-12 MHz.

A receive beamformer selects the relative delays that control the orientation of the receive beam with respect to the transducer array. In this way, the ultrasound system acquires echo data from a series of focal points to form an image that maps different tissue structures in the body. B-mode ultrasound imaging is one category of imaging techniques for generating such two-dimensional images. In various contexts, B-mode imaging may also be referred to by other names, such as intensity or 2D-imaging.

Doppler ultrasound imaging systems have been used to determine the blood pressure and the blood flow within the heart and the vascular system. Blood creates a relatively small echo signal as compared with the surrounding stationary tissue. By discriminatively detecting frequency-shifted echoes, an ultrasound imaging system can selectively detect the motion of blood and thereby form an image of vascular pathways or other fluid. Color-mode ultrasound imaging is a category of imaging techniques for generating such two-dimensional images. This category of techniques is also known by other nomenclature, such as Doppler-mode imaging, C-mode imaging, color flow mapping, and flow-mode imaging.

A third category of imaging techniques is known as spectral Doppler-mode imaging, in which techniques are used to perform spectral analysis over time to characterize the behavior of flows and other motions at a point. This category of techniques is also known by other nomenclature, such as the analysis of range-gate locations and color-spectrum imaging.

Other categories of ultrasound imaging are also known, as well as combinations thereof. A duplex-mode imaging system may alternatively measure and generates two modes of images. For example, a duplex-mode system may interleave B-mode images with color-mode images. By displaying a combination or interleaving of such images, a duplex-mode imaging system may provide an enhanced image with both B-mode and Doppler information to an operator.

Similarly, a triplex-mode imaging system may alternatively measure and generates three modes of images. For example, a triplex-mode system may interleave B-mode images with color-mode images and spectral Doppler-mode images. Higher numbers of interleaved modes are also possible.

The various imaging modes generally have different voltage requirements. One design factor is that, in general, higher voltage levels for the ultrasound beams allow the system to create images with higher axial resolution, better far-field penetration, and other qualities. This factor is tempered by safety considerations: too much power can be unhealthy or damaging to the body being imaged. Thus, another design factor affecting the choice of voltage levels is safety limits, such as the transducer thermal limit. In general, ultrasound systems are designed to balance these considerations by using a high enough voltage level while ensuring that that the energy or time-averaged power deposited into a body is limited to a safe level. The balancing point of these considerations depends on the type of transducer being used, and other equipment-related factors. In addition, this balancing point for the appropriate voltage level also depends on the type of imaging mode being employed by the equipment.

Different imaging modes typically use different pulse profiles for the ultrasound power being transmitted into the body. The pulse profiles indicate the variation over time of the transmitted ultrasound power. The duration of these various pulse profiles constrains the peak voltage levels that can be used in the pulses. In general, pulses with shorter-duration profiles may be created with high peak voltages, while pulses with longer-duration profiles are created with lower peak voltages to ensure that the total energy used in the pulses remains within a safe limit.

For example, it may be desirable to create a B-mode image using a broadband burst, which may be achieved with a short-duration or single-cycle burst. In contrast, it may be desirable to create a color-mode image using a narrow-band burst, which may be achieved with a long-duration series of several cycles. As a result, the B-mode burst may be operated at a higher voltage than the color-mode burst. This difference in voltage levels optimizes the image quality while keeping both bursts within safe limits on their total energy.

Because the different imaging modes may operate with different levels of voltage while maintaining safety standards, duplex and triplex ultrasound systems use multiple voltage supplies for the various imaging modes. For example, a high-voltage supply, such as a 100 V supply, may be used to power the transducer for a B-mode image. The system may then quickly switch to a lower-voltage supply, such as a 40 V supply for the transmit voltage for a color-mode image. This switching between different voltage supplies allows the system (1) to rapidly acquire images from the different modes, interleaving them into a smooth display for the benefit of a user, and (2) to operate in each mode with an optimal voltage supply that allows a maximum but safe voltage level for that imaging mode. However, the multiple voltage supplies and the switching between voltage supplies add cost and complexity to ultrasound systems.

Portability is becoming an increasingly desirable feature in ultrasound imaging systems. Systems with simplified hardware better lend themselves to portable designs.

### BRIEF SUMMARY

The present invention is defined by the following claims, and nothing in this section should be taken as a limitation on those claims. By way of introduction, the preferred embodiments described below include methods and transmitters for transmitting and receiving ultrasound pulses with duty cycles and pulse profiles that reflect energy constraints as well as the performance requirements of various imaging modes.

Described herein are systems and methods for performing duplex-mode, triplex-mode, or other multi-mode imaging in ultrasound systems. One example of a method for generating an ultrasound image involves duplex or multimode operation of an ultrasound system. The system alternates between two or more modes of operation, interleaving images from the several modes to generate a combined image. The system may be simplified by sharing a single voltage supply among two or more of the image modes. The imaging modes may be appropriately modified so that they can be operated with ultrasound pulses at a single shared voltage level.

In one implementation, this example method includes steps of generating transmit pulses at a predetermined voltage level for the first imaging mode, acquiring a first image, generating transmit pulses at the predetermined voltage level for the second imaging mode, and acquiring a second image. Images from the two modes may then be combined into an interleaved image.

The first and second imaging modes may be, for example, a Doppler-spectral ultrasound imaging mode and a B-mode ultrasound imaging mode. To accommodate a generally higher voltage level preferred for B-mode imaging, the Doppler-spectral mode may use pulse trains with a reduced duty cycle. For example, the duty cycle may be reduced to satisfy a restriction on the surface temperature of a transducer. Alternatively, or in addition, the duty cycle may be reduced to satisfy a restriction on the output power of a transducer.

A variety of techniques are also disclosed for enhancing image quality ultrasound imaging, such as increasing the voltage level for color-mode and spectral Doppler-mode pulse profiles, shortening the transmit duty cycle for color-mode and spectral Doppler-mode pulse profiles, increasing the voltage level for B-mode pulse profiles, increasing the number of transmit cycles for B-mode pulse profiles, decreasing the transmit frequency B-mode pulses, and modifying the B-mode dynamic filter settings, among others. In some implementations of an ultrasound imaging system, multimode imaging may be performed using a single fixed-voltage power supply. In some implementations of an ultrasound imaging system, multimode imaging may be performed using a number of fixed-voltage power supplies that is less than the number of modes used for multimode imaging.

### BRIEF DESCRIPTION OF SEVERAL VIEWS OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound transmitter having a single fixed-voltage power supply.

FIG. 2 illustrates one implementation of temporal pulse profiles for unipolar color-mode ultrasound pulses.

FIG. 3 illustrates one implementation of temporal pulse profiles for bipolar color-mode ultrasound pulses.

FIG. 4 is a block diagram of one embodiment of an ultrasound transmitter having a plurality of fixed-voltage power supplies.

FIG. 5 is a flow diagram illustrating one implementation of a procedure for duplex imaging.

FIG. 6 is a flow diagram illustrating one implementation of a procedure for modifying an ultrasound imaging system.

### DETAILED DESCRIPTION OF THE PREFERRED EMBODIMENTS

One of the challenges facing designers of ultrasound imaging systems is the task of using a single fixed-voltage power supply in multi-mode systems. Duplex-mode systems may effectively offer the user the ability to see two images acquired in different operating modes overlaid together into a single image. For example, a duplex-mode system may present a combined image of B-mode and color-mode images, overlaid together to provide enhanced information for the user. Similarly, a triplex-mode system may provide combined views based on B-mode, color-mode, and spectral Doppler-mode images. Since the various modes generally have different pulse profiles, previous systems have optimized performance by operating the different modes at different peak power levels. These different peak power levels have required multiple power supplies for the multiple operating modes.

Each additional power supply and the switching between power supplies add cost and complexity to an ultrasound system. One cost-saving approach would be to use a single fixed-voltage power supply, or fewer supplies than the number of different operating modes, and then to accept different performance in one or more of the operating modes. For example, using shorter color-mode or spectral Doppler-mode pulse profiles may reduce the accuracy and sensitivity of velocity estimation in these operation modes.

Another approach would be to switch the voltage level as needed when the system switches between different operating modes. However, switching the voltage level of a voltage supply could introduce switching noise into the system. Switching the voltage level of the voltage supply slowly may not help: noise-free switching takes time, and could lead to undesirable delays between acquisitions of images from different modes.

Alternatively, a duplex-mode or triplex-mode system with only one power supply might avoid switching its voltage levels. In this situation, the B-mode, color-mode, spectral Doppler-mode, or other modes of imaging operate with the same voltage level. The results may be different because the bandwidth limit on the color-mode or spectral Doppler-mode hinders the signal level available for the B-mode image.

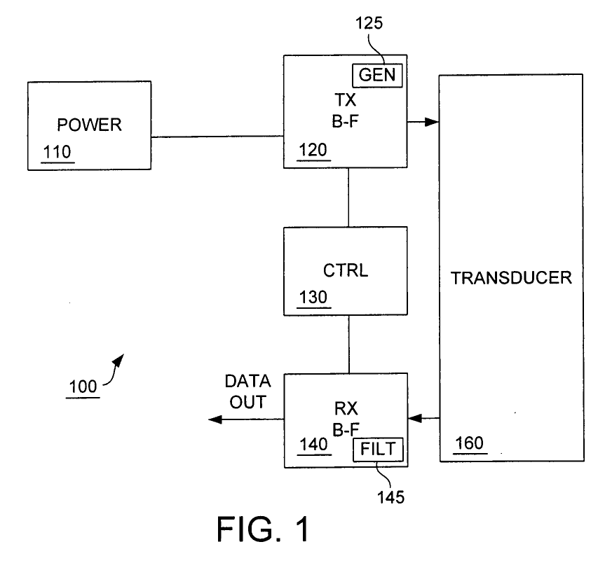

FIG. 1 illustrates a multi-mode ultrasound system 100 that uses modified duty cycles for transmit pulses to enable the system 100 to work with a single power supply. The multi-mode ultrasound system may be a duplex-mode system or triplex-mode system, or other system that allows interleaving of multiple image modes. The multi-mode ultrasound system 100 includes a fixed-voltage power supply 110, a transmit beamformer 120 with a waveform generator 125, a control unit 130, a receive beamformer 140 with a dynamic receive filter 145, and a transducer 160.

The transmit beamformer 120 has a plurality of channels. Each channel has a waveform generator 125, a delay, and an amplifier for generating acoustic transmit beams from relatively delayed and apodized waveforms.

The transducer 160 is a one- or two-dimensional array of capacitive membrane or piezoelectric elements. The transducer 160 transmits a pulse of ultrasound into a body being studied, such as a patient's body (not shown) being examined during a medical assessment or treatment in response to signals from the transmit beamformer 120. The transmitted ultrasound signals may be square pulses or sinusoidal pulses, or may have other pulse shapes, and may be transmitted with one cycle or several cycles, as appropriate for the operating mode of the ultrasound system. The cycles are generally created with cycle frequencies in the range of 2-12 MHz, such as 2.5, 3.5, 5, 6.5, 7.5, 10, or 12 MHz, or other frequencies. Ultrasound reflected from the patient's body is then detected by the transducer 160, and is used by the system 100 to image structures within the body.

The receive beamformer 140 has a plurality of processing channels with compensating delay elements connected to a summer. The receive beamformer uses a delay value for each channel to collect echoes reflected from a selected focal point. Consequently, when delayed signals are summed, a strong signal is produced corresponding to this point. The receive beamformer 140 processes the electrical signals received from the transducer 160 and generates data to be output for further signal processing and display. When the system 100 operates in duplex or triplex modes, the output data may be combined and displayed together as an overlay image for the benefit of an operator. The transmit and receive beamformers 120 and 140 operate under the control of the control unit 130. For example, in response to an operator's input, the control unit 130 may select whether the beamformers 120 and 140 operate in simple B-mode, or in duplex B-mode/color-mode, or in triplex B-mode/color-mode/spectral Doppler-mode.

The waveform generator 125 creates the transmit profile—the temporal profile of the ultrasound pulse to be transmitted into the body. The ultrasound pulse may be formed as a train of pulses, each with a predetermined profile. If each pulse is a square pulse, then the pulse train may be formed by switching circuits that alternately connect and disconnect a flow of power between a power supply and the transducer.

The waveform generator 125 may be implemented using, for example, a MFET network with a fixed or programmable set of waveforms. The waveform generator 125 may be implemented, for example, as a hardware filter based on an application-specific integrated circuit (ASIC), a digital signal processor (DSP), a field-programmable gate array (FPGA), a microprocessor, a microcontroller, or any other appropriate circuitry currently known or hereafter developed. These hardware components may be operated under the control of appropriate firmware or software.

The waveform generator 125 determines the transmit profile under instructions from the control unit 130, and may generate qualitatively different pulse profiles for different operating modes such as B-mode, color mode, harmonic mode, tissue harmonic mode, contrast agent mode, and spectral Doppler-mode operation, among others.

The waveform generator 125 may be an on-off generator, which generates unipolar pulse profiles with two values: maximum amplitude (on) and zero amplitude (off). Alternatively, the waveform generator 125 may be an tri-mode generator, which generates bipolar pulse profiles with three values: maximum positive amplitude (+), maximum negative amplitude (−), and zero amplitude (off). Alternatively, the waveform generator 125 may generate more general pulse profiles, including a variety of amplitude levels or a continuum of amplitude levels.

The dynamic receive filter 145 filters the ultrasonic signals received from the body through the transducer 160. The dynamic receive filter 145 may be adaptable based on the nature of the signals received. The dynamic receive filter 145 may be a low-pass filter that processes baseband signals, or a bandpass filter that processes RF signals, or may have a more complex filter design. The dynamic receive filter 145 may be implemented, for example, as a hardware filter based on an ASIC, a DSP, an FPGA, a microprocessor, a microcontroller, or any other appropriate circuitry currently known or hereafter developed. These hardware components may be operated under the control of appropriate firmware or software. The dynamic receive filter 145 may include two or more separate hardware units, such as one for filtering received B-mode pulses, and one for filtering received color-mode pulses.

The dynamic receive filter 145 may be modified so that it can suitably process received B-mode ultrasound signals at more than one center frequency. This modification may be useful for implementations of the system 100 in which B-mode signals are transmitted at a high frequency during simple (non-duplex) B-mode operation, and are transmitted at a low frequency during duplex operation.

The power supply 110 provides power to the transmit beamformer 120 to generate the ultrasound transmitted into the body. Various implementations may be used for the power supply, such as subsystems that use transformers, power transistors, and other tools for generating reliable voltage supplies. As depicted in the system 100, the power supply 110 may be coupled to the transmit beamformer 120, and may provide power to the transducer 160 through the transmit beamformer 120. Alternatively, the power supply 110 may be directly coupled to the transducer 160, and may provide power directly to the transducer 160. The power supply 110 may provide fixed-voltage electrical power, and the maximum voltage level generated by the transducer may be limited by the voltage of the power supply 110.

The power supply 110 may be selected to provide an adequate voltage level, or instantaneous power level, for creating short-duration imaging pulses (such as B-mode pulses). When the system 100 operates in a mode using longer-duration pulses (such as in color-mode or spectral Doppler-mode), the average power delivered to the body is regulated by the waveform generator 125. Rather than reducing the voltage level, the waveform generator 125 may avoid excessive power transmission by reducing the duty cycle for longer-duration pulses.

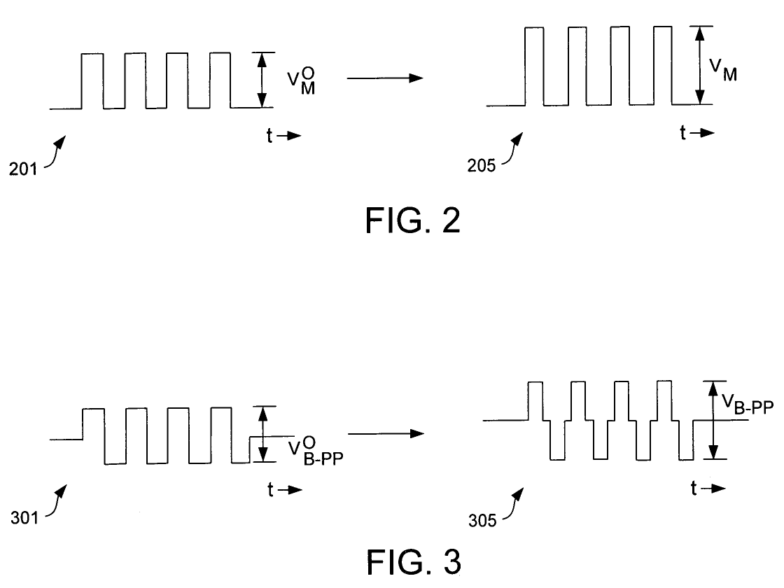

FIG. 2 illustrates two examples of unipolar pulse profiles 201 and 205 that provide limited average power levels in the color-mode operation of an ultrasound system. The profiles are shown as temporal graphs, with time (t) on the abscissa and pulse amplitude on the ordinate. The pulse profiles 201 and 205 are both suitable for color-mode operation in a duplex mode, and can be interleaved with pulse profiles designed for B-mode operation. In other embodiments, fewer or more cycles are used. The two profiles 201 and 205 illustrate different approaches to limiting the time-averaged power delivered to a body. The pulse profile 201 uses a reduced amplitude to limit the time-averaged power. In contrast, pulse profile 205 uses a reduced duty cycle to limit the time-averaged power, thereby allowing a greater amplitude for the pulse profile.

Pulse profile 201 illustrates a pulse with a relatively long durations: a color-mode pulse with four cycles of ultrasound energy. Each cycle is a constant-amplitude period of transmitted ultrasound, with an amplitude V_{M}°. The amplitude V_{M}° may correspond, for example, to a supply voltage of 40 V (depending on the efficiency, impedance, and other characteristics of the transducer). The amplitude V_{M}° may have a lower amplitude than the amplitude of a short-duration pulse (such as a B-mode pulse, not shown) in order to ensure that the total power transmitted during each pulse does not exceed desired limits. The duty cycle of pulse profile 201 is 50%, meaning that the high-amplitude transmission occurs during 50% of the duration of the pulse profile. The duration of each cycle in pulse profile 201 and the duty cycle of pulse profile 201 may be chosen to optimize image quality.

The number of cycles in a color-mode pulse profile is selected based on various criteria, such as the desire to use a pulse with a narrow band of frequency components. In a variety of situations, the number of transmit cycles is usually at least 4 to provide the appropriate narrow band frequency spectrum. If the number of cycles is less than 4, the color-mode's velocity estimation may become less accurate and sensitivity may be reduced. This observation tends to preclude designers from raising the color-mode transmit voltage by reducing the number of cycles. However, the transmit voltage may be increased by reducing the duty cycle of the transmit pulse.

Pulse profile 205 illustrates another color-mode pulse with four cycles of ultrasound energy. Each cycle is a constant-amplitude period of transmitted ultrasound, but with an amplitude V_{M} that is greater than V_{M}°. The amplitude V_{M} may correspond, for example, to a supply voltage of 60 V (again, depending on the efficiency, impedance, and other characteristics of the transducer). The amplitude V_{M} is selected in response to considerations of design simplicity: it is close to or equal to the amplitude of a short-duration pulse (such as a B-mode pulse, not shown) in order to allow an ultrasound system to operate with a single constant-voltage power supply for both B-mode and color-mode imaging. The duty cycle of pulse profile 205 is 33%, which is less than the duty cycle for pulse profile 201.

The reduced duty cycle in pulse profile 205 compensates for the increased amplitude of pulse profile 205. The duration of each cycle in pulse profile 205 or the duty cycle of pulse profile 205 may be chosen to limit power supply switching. The duration and duty cycle may be determined, for example, from computations or measurements of the total transmitted power or of the resulting temperature of the transducer, or other criteria. In some implementations of pulse profile 205, image quality may be partially sacrificed so that the ultrasound system may operate with amplitude V_{M}.

The values noted above indicate a 50% increase in voltage supply and a 33% reduction in duty cycle when comparing pulse profile 205 to pulse profile 201 (60 V as compared to 40 V; 33% duty cycle as compared to 50% duty cycle). Other implementations of pulse profile 205 are also possible. For example, pulse profile 205 may represent an increase in voltage supply of 0% to 150% (such as, for example, a 0%, 20%, 40%, 60%, 80%, 100%, 125%, or 150% increase) in voltage supply, and a decrease in duty cycle of 5% to 80% (such as, for example, a 5%, 10%, 20%, 25%, 30%, 40%, 50%, 60%, 70%, 75%, or 80% decrease).

FIG. 3 illustrates two examples of bipolar pulse profiles that provide limited average power levels in the color-mode operation of an ultrasound system. As with the pulse profiles from FIG. 2, pulse profiles 301 and 305 are both suitable for color-mode operation in a duplex mode, and can be interleaved with pulse profiles designed for B-mode operation. In a manner similar to that discussed above, pulse profile 301 uses a reduced amplitude to limit the time-averaged power, while pulse profile 305 uses a reduced duty cycle to limit the time-averaged power. Bipolar pulse profile 301 has a peak-to-peak amplitude of V_{B-PP}°, while bipolar pulse profile 301 has a larger peak-to-peak amplitude of V_{B-PP} and a smaller duty cycle.

The approach of using a reduced duty cycle, as illustrated by pulse profiles 205 and 305, allows a multi-mode ultrasound system to be designed with fewer power supplies. With the color-mode imaging now adapted for use with higher-voltage electrical power, the system uses the same power supply for both B-mode imaging and color-mode imaging. This adaptation thus eliminates the need for a separate lower-voltage power supply for color-mode imaging.

The same approach can also be used for adapting other imaging modes to share power supplies. The B-mode imaging discussed herein is representative of operating modes that generally have short-duration transmit pulses. Similarly, the color-mode imaging discussed above is representative of operating modes that generally have long-duration transmit pulses, such as spectral-Doppler imaging modes. With long-duration modes being operated at higher-voltage electrical power and shorter duty cycles, these modes may make use of the one or more power supplies that are already needed for short-durations modes.

Using a single fixed-voltage power supply for two or more imaging modes may provide a significant savings in cost or design complexity. This multitasking of power supplies involves operating the imaging modes at substantially the same voltage level. With the power level fixed or held constant, the duty cycle in at least one of the imaging modes is selected to ensure that an appropriate amount of energy is delivered in that mode. For example, by reducing the transmit duty cycle for color-mode pulses at duplex or triplex mode, a higher voltage may be used for duplex-mode operation, thereby enhancing the performance of B-mode imaging.

For example, an ultrasound imaging system may operate in a variety of settings, including a pure B-mode setting, and a duplex-mode setting that interleaves B-mode and color-mode images. While operating in the pure B-mode setting, the system may adjust the power supply to a high voltage (such as 100 V, for example) that is optimal for B-mode pulses. When an operator switches the system to operate in duplex mode, a system without a reduced duty cycle may adjust the power supply to a low voltage (such as 40 V, for example) that is appropriate for the average-power limits in color-mode operation, but which may be detrimental to B-mode performance.

However, by using a reduced duty cycle for the color-mode pulse profile, a system may be designed to select a high voltage (such as 100 V, for example) for the B-mode setting, and an intermediate voltage (such as 60 V, for example) for the duplex-mode setting. The shortened duty cycle allows an increased voltage (60 V instead of 40 V, for example) and thus a greater instantaneous power to be used while keeping within the average power limits for the color-mode operation. The increased voltage enhances the performance of the B-mode imaging during duplex operation.

Additional techniques may also be employed to enhance the B-mode images during duplex operation. For example, the number of B-mode cycles may be increased, such as from one or two cycles for single-mode (non-duplex) B-mode operation, up to two or three cycles for B-mode imaging in duplex mode. In B-mode imaging, axial and lateral image resolution tends to be an important factor. A wide-band transmit pulse is used for B-mode imaging, so the number of transmit cycles may be limited to one or two cycle per transmit pulse. However, when the system operates in duplex or triplex mode, the color-mode or spectral Doppler-mode component of the final image may be the most important imaging mode. In this situation, increasing the number of B-mode cycles may produce an acceptable reduction in axial or lateral resolution for the B-mode component of the duplex or triplex image. Thus, the number of transmit cycles per B-mode pulse may be increased. This increase may provide a better B-mode signal to noise ratio.

Another technique for tempering compromises in the quality of the B-mode images involves lowering the transmit frequency for the B-mode pulses when operating in duplex or triplex mode. Reducing the transmit frequency may improve the signal to noise ratio of the received B-mode signals, and may thus be another tool for dealing with compromises in the quality of B-mode images.

The fixed-voltage power supply may be configured with some degree of flexibility in the voltage. For example, the fixed-voltage power supply may be configured to change its fixed voltage when the system switches between operating modes. Thus, the power supply may provide, for example, (a) a fixed voltage of 60 V when the system is operating in duplex or triplex mode (with line-groups of lines-, or frame-interleaved B-mode and color mode and/or spectral Doppler mode measurements), (b) a fixed voltage of 100 V when the system is operating in simple B-mode (without color mode and/or spectral Doppler mode imaging), and (c) a fixed voltage of 40 V when the system is operating in simple color mode or spectral Doppler mode (without B-mode imaging).

Additionally, the system may be equipped with a fixed-voltage power supply capable of making small but rapid and substantially noise-free changes in its voltage level. In this case, the supply voltage may be switched as the system rapidly alternates between operating modes during duplex or triplex operation. The power supply may be used for more than one imaging mode while allowing some variation between the voltages used for each of the operating modes. For example, if the power supply can rapidly and noiselessly switch from 55 V to 65 V, then this power supply may be used during duplex operation to supply power at 55 V for color-mode pulses and at 65 V for B-mode pulses. Thus, the B-mode pulses would not need to be supplied with exactly the same voltage as the color-mode pulses. Rather, the B-mode could be powered with a voltage level that is close to (within 10 V or 18% of) the voltage for the color-mode pulses. Depending on the capabilities of the power supply, the system may be designed so that the B-mode and color-mode pulses need not be exactly matched in voltage level, but may be close-so that the B-mode pulses are within 2%, 5%, 10%, 25%, 30%, 40%, or 50%, or 75% of the voltage level of the color-mode pulses. This close but not exact matching may alleviate some of the other design constraints discussed above.

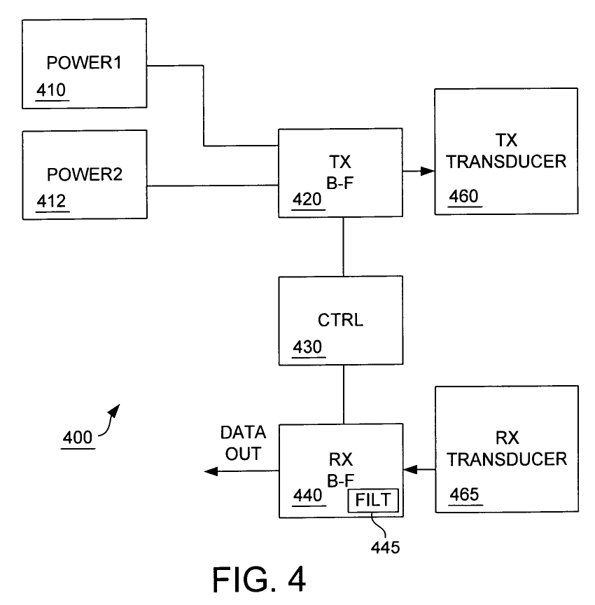

FIG. 4 illustrates a multi-mode ultrasound system 400 that uses modified duty cycles for transmit pulses to enable the system 400 to work with more than one power supply. The multi-mode ultrasound system 400 includes two fixed-voltage power supplies 410 and 412, a transmit beamformer 420, a control unit 430, a receive beamformer 440, a transmit transducer 460, and a receive transducer 465, which operate in a manner similar to the power supply 110, transmit beamformer 120, control unit 130, receive beamformer 140, and transducer 160 of the previously described system 100 shown in FIG. 1. Here, the transmission and reception components of the transducer are indicated in separate units, the transmit transducer 460, and the receive transducer 465, illustrating that the transducer components may be implemented in separate units as appropriate to some implementations of the system.

System 400 uses the two fixed-voltage power supplies 410 and 412 to supply power for a multimode operation with three or more ultrasound imaging modes. System 400 may use the techniques described above, such as reduced duty cycles, reduced cycle durations, reduced transmission frequencies, or increased numbers of cycles per pulse, so that one of the fixed-voltage power supplies 410 and 412 may be used to provide power to more than one of the imaging modes. More generally, variations of system 400 may also use these techniques so that they use different numbers of power supplies Np to implement multimode operation with different numbers of operating modes Nm, while keeping Np less than Nm.

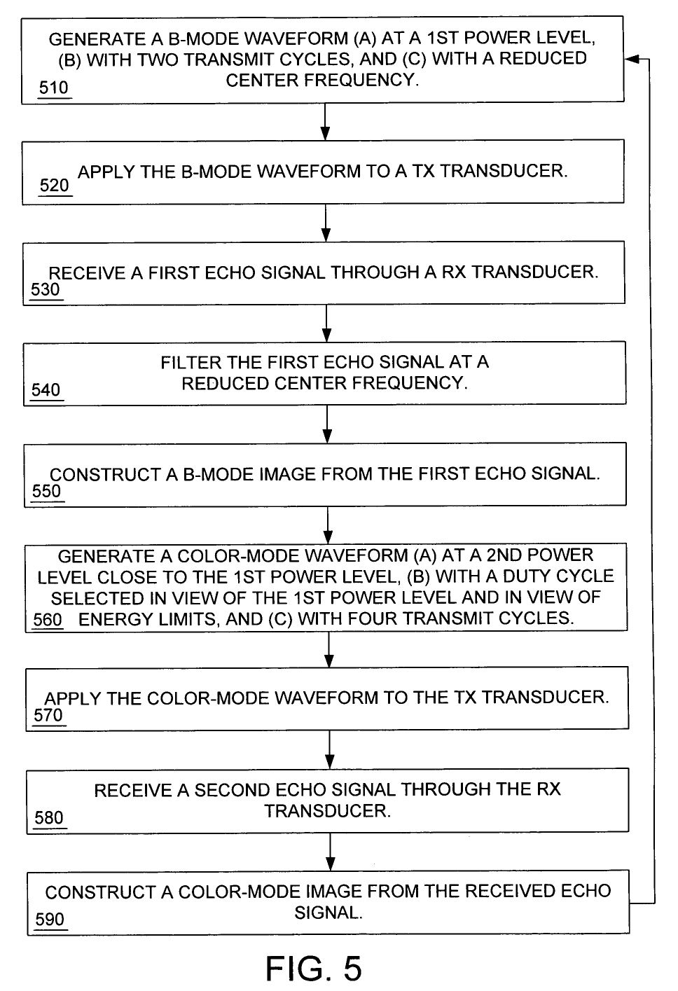

FIG. 5 illustrates one embodiment of a procedure for performing duplex ultrasound imaging using B-mode and color-mode imaging. The procedure begins in act 510 by generating a B-mode waveform for duplex operation using power supplied at some power level, denoted as the first power level. In some versions of the procedure, the first power level used for the B-mode during duplex imaging may be lower than a voltage level that would be used for single-mode (non-duplex) B-mode imaging. To enhance the signal level of the received B-mode ultrasound, the B-mode waveform may have an increased number of transmit cycles, such as two transmit cycles. As an additional or alternative enhancement, the B-mode waveform may specify a reduced transmit frequency during duplex operation, such as 3 MHz, or 5 MHz, or 6 MHz, for example (instead of values such as 7 MHz, 7.5 MHz or 10 MHz, for example, during non-duplex, single-mode B-mode operation).

In act 520, the B-mode waveform generated in act 510 is applied to a transducer. The transducer sends an ultrasound signal into a body under examination. A first ultrasound echo signal from the body is received by a receiving transducer in act 530. Depending on the system implementation, the receiving transducer in act 530 may be the same as or different from the transmitting transducer in act 520. In act 540, the ultrasound signal received in act 530 may be filtered, for example by a fixed filter or by a dynamic filter, with signal processing techniques optimized for the received ultrasound signals. The filtering and any other processing in act 540 may be tailored to a reduced transmit frequency used for the duplex B-mode waveform transmitted in act 510. In act 550, a B-mode image is constructed from the ultrasound signal received in act 530.

A color-mode pulse waveform for duplex operation is generated in act 560. The color-mode waveform defines an ultrasound pulse with a second power level that is close to or the same as the first power level that was used in act 510. For example, the second power level used for the duplex color-mode waveform may be within 2%, 10%, 25%, or 50%, of the first power level, or otherwise close to the first power level. In some versions of the procedure, the second power level used for the color-mode during duplex imaging may be greater than a voltage level than would be used for single-mode (non-duplex) color-mode imaging. The color-mode waveform may be prepared with a reduced duty cycle, in view of the second power level being used for the duplex color-mode waveform. The color-mode waveform may have a typical number of transmit cycles for a color-mode waveform, such as four transmit cycles. Alternatively, the color-mode waveform may have a larger or smaller number of transmit cycles, as appropriate for the system implementation in which the procedure is implemented.

In act 570, the color-mode waveform generated in act 550 is applied to a transducer, which then sends an ultrasound signal into the body under examination. A second ultrasound echo signals from the body may then be received by the transducer and processed to create a color-mode image in act 580.

In act 590, a color-mode image is constructed from the ultrasound signal received in act 580. The procedure may be repeated to sequentially provide interleaved or combined B-mode and color-mode data for a real-time duplex-mode image. Acts 510-550 may be performed once for a single scan line or repeated for a plurality of scan lines before beginning act 560. Single-line, groups of lines, frames, and groups of frames interleaving may be used for switching between modes in duplex operation.

The procedure outlined in FIG. 5 describes duplex imaging using two modes: B-mode and color-mode imaging. The procedure may be adapted for other duplex operations, such as B-mode and spectral Doppler-mode imaging, or to triplex operations, such as B-mode, color-mode, and spectral Doppler-mode imaging. The procedure may also be adapted to other multi-mode operations.

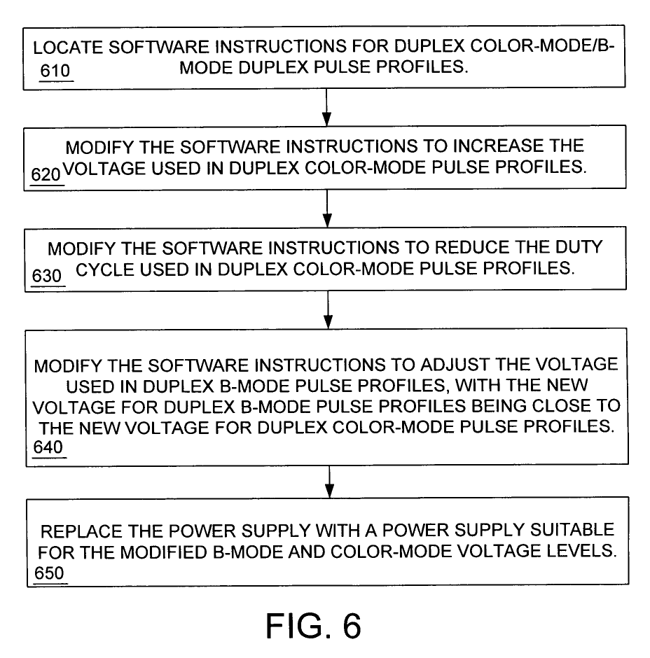

FIG. 6 describes a procedure for retrofitting or upgrading ultrasound diagnostic equipment to enhance duplex-mode, triplex-mode, or other multimode operation. The procedure may be implemented by altering hardware in the system, by replacing hardware in the system, by altering software in the system, by replacing software in the system, or by any combination of these actions, as appropriate for the original configuration of the ultrasound system. These procedures may be implemented either by individually modifying ultrasound systems, or by preparing a software patch that may then be applied to one or more ultrasound systems. For illustrative purposes, FIG. 6 describes the procedure in the context of modifying software in an ultrasound system that has a duplex mode for combining B-mode and color-mode imaging. The procedure may be adapted for other configurations of hardware and software, and to other configurations of multimode imaging.

In act 610, the software instructions or reference data that implement or define pulse profiles for duplex-mode, triplex-mode, or other multi-mode operation are located in the ultrasound system. In act 620, the software instructions or reference data are edited, replaced, or otherwise modified to increase the voltage used in color-mode pulse profiles during duplex-mode, triplex-mode, or other multi-mode operation. In act 630, the software instructions or reference data are edited, replaced, or otherwise modified to reduce the duty cycle used in color-mode pulse profiles during duplex-mode, triplex-mode, or other multi-mode operation.

In act 640, the software instructions or reference data are edited, replaced, or otherwise modified to reduce the voltage used in B-mode pulse profiles duplex-mode, triplex-mode, or other multi-mode operation. The new replacement value for B-mode voltage may be close to or substantially the same as the new replacement value for color-mode voltage duplex-mode, triplex-mode, or other multi-mode operation.

In act 650, the system's power supply may be modified or replaced so that duplex-mode, triplex-mode, or other multi-mode operation can share a power supply for both B-mode and color-mode imaging.

While the invention has been described above by reference to various embodiments, it should be understood that many changes and modifications can be made without departing from the scope of the invention. It is therefore intended that the foregoing detailed description be understood as an illustration of the presently preferred embodiments of the invention, and not as a definition of the invention. It is only the following claims, including all equivalents, which are intended to define the scope of this invention.

1. A method for combining a first imaging mode and a second imaging mode to generate a combined ultrasound image, the method comprising:
(a) generating transmit pulses at a predetermined voltage level for the first imaging mode;
(b) acquiring a first image in the first imaging mode in response to the transmit pulses generated in act (a);
(c) generating transmit pulses at the predetermined voltage level for the second imaging mode, with a duty cycle selected in response to one or more of: a restriction on surface temperature of a transducer, and a restriction on transducer power output;
(d) acquiring a second image in the second imaging mode in response to the transmit pulses generated in act (c); and
(e) displaying the first and second images.

2. The method of claim 1, where said generating transmit pulses at the predetermined voltage level for the first imaging mode comprises on-off switching of a single DC voltage supply.

3. The method of claim 1, where the first imaging mode is a B-mode ultrasound imaging mode.

4. The method of claim 1, where the second imaging mode is a Doppler-spectral ultrasound imaging mode.

5. The method of claim 1, where the second imaging mode is a color-mode ultrasound imaging mode.

6. The method of claim 1, where acts (a)-(e) are repeated to generate an interleaved sequence of first and second images.

7. The method of claim 1, where the duty cycle is selected to meet a maximum transducer surface temperature limit of 43° C.

8. The method of claim 1, where the duty cycle is selected to meet a maximum transducer surface temperature limit of 41° C.

9. The method of claim 1, where the duty cycle is selected to meet a maximum transducer surface temperature limit of 50° C.

10. The method of claim 1, where the duty cycle is selected to meet a maximum transducer surface temperature limit in the range of 38° C. to 40° C.

11. The method of claim 1, where the duty cycle is selected to meet a maximum transducer surface temperature limit in the range of 35° C. to 38° C.

12. The method of claim 1, where the duty cycle is selected to meet a maximum transducer surface temperature limit specified by International Standard IEC 60601-2-37.

13. The method of claim 1, where the duty cycle is selected to meet a maximum transducer power output of 720 mW/cm².

14. The method of claim 1, where the duty cycle is selected to meet a maximum transducer power output in the range of 550 to 600 mW/cm².

15. The method of claim 1, where the duty cycle is selected to meet a maximum transducer power output in the range of 575 to 650 mW/cm².

16. The method of claim 1, where the duty cycle is selected to meet a maximum transducer power output in the range of 550 to 700 mW/cm².

17. The method of claim 1, where the duty cycle is selected to meet a maximum transducer power output of 50 mW/cm².

18. The method of claim 1, where the duty cycle is selected to meet a maximum transducer power output in the range of 45 to 48 mW/cm².

19. The method of claim 1, where the duty cycle is selected to meet a maximum transducer power output in the range of 44 to 46 mW/cm².

20. The method of claim 1, where the duty cycle is selected to meet a maximum transducer power output specified by “Information for manufacturers Seeking Marketing Clearance of Diagnostic Ultrasound Systems and Transducers.”

21. The method of claim 1, where act (a) includes transmitting a train of unipolar pulses.

22. The method of claim 1, where act (a) includes transmitting a train of bipolar pulses.

23. The method of claim 1, where act (a) comprises
(a1) setting a voltage supply to the predetermined voltage level; and
(a2) using one or more switches to engage or disengage the voltage supply.

24. The method of claim 1, where the predetermined voltage level is a user-selectable voltage level.

25. The method of claim 1, where the predetermined voltage level is selected in response to one or more of: the restriction on surface temperature of the transducer, and the restriction on transducer power output.

26. The method of claim 1, where the predetermined voltage level is selected to meet a maximum transducer surface temperature limit of 43° C.

27. The method of claim 1, where the predetermined voltage level is selected to meet a maximum transducer power output in the range of 550 to 600 mW/cm².

28. A transmit method for ultrasound imaging, the method comprising:
(a) generating a first pulse train for a first mode of operation at a fixed voltage level selected in response to a restriction on surface temperature of a transducer;
(b) applying the first pulse train to a transducer;
(c) generating a second pulse train for a second mode of operation substantially at the fixed voltage level, wherein the second mode of operation is different from the first mode of operation;
(d) applying the second pulse train to the transducer.

29. The method of claim 28 wherein act (c) comprises generating the second pulse train with pulses having a duty cycle chosen in response to the restriction on surface temperature of the transducer.

30. The method of claim 28 wherein the first pulse train is a B-mode pulse train.

31. The method of claim 28 wherein the second pulse train is a color-mode pulse train.

32. The method of claim 28 wherein the first pulse train is a B-mode pulse train and the second pulse train is one of: a color-mode pulse train and a spectral Doppler-mode pulse train.

33. The method of claim 28 wherein the first and second pulse trains are unipolar pulse trains.

34. The method of claim 28 wherein the first and second pulse trains are bipolar pulse trains.

35. The method of claim 28 wherein the first pulse train includes at least one transmit cycle and wherein the second pulse train includes at least four transmit cycles.

36. The method of claim 28 wherein the first pulse train includes at least two transmit cycles and wherein the second pulse train includes at least four transmit cycles.

37. The method of claim 28 wherein the first pulse train is a B-mode pulse train that includes at least two transmit cycles, and wherein the second pulse train is a color-mode pulse train that includes at least four transmit cycles.

38. The method of claim 28, further comprising:
(e) detecting a received pulse train from the transducer;
(f) filtering the received pulse train with a lower center frequency than a center frequency of the transmit pulse train.

39. An ultrasound transmitter comprising:
a pulse train generator operable to generate a first pulse train for a first mode of operation and a second pulse train for a second mode of operation;
wherein the second mode of operation is different from the first mode of operation;
a transducer coupled to the pulse train generator; and
a power supply coupled to the transducer and operable to supply a fixed voltage level to the transducer for both the first and second pulse trains;
where the fixed voltage level is selected in response to one or more of: a restriction on surface temperature of the transducer, and a restriction on transducer power output.

40. The transmitter of claim 39, where the second mode of operation is configured to transmit pulses with a duty cycle, where the duty cycle is selected in response to one or more of: the restriction on surface temperature of the transducer, and the restriction on transducer power output.

41. The transmitter of claim 39 wherein the pulse train generator comprises a bipolar pulse train generator.

42. The transmitter of claim 39 wherein the first pulse train is a B-mode pulse train and the second pulse train is a color-mode pulse train.

43. The transmitter of claim 39 wherein the first pulse train is a B-mode pulse train and the second pulse train is a spectral Doppler-mode pulse train.

44. The transmitter of claim 39 wherein the first pulse train includes at least one transmit cycle and wherein the second pulse train includes at least four transmit cycles.

45. The transmitter of claim 39 wherein the first pulse train includes at least two transmit cycles and wherein the second pulse train includes at least four transmit cycles.

46. An ultrasound transmitter comprising:
a number Np of fixed-voltage power sources;
a pulse train generator coupled to the number of fixed-voltage supplies;
wherein the pulse train generator is operable to generate pulse trains for a number Nm of operating modes, and wherein the number of operating modes include at least two of: B-mode operation, color-mode operation, and spectral Doppler-mode operation;
wherein at least one of the operating modes has a pulse profile with a duty cycle selected in response to one or more of: a restriction on surface temperature of the transducer, and a restriction on transducer power output;
wherein the number Nm of operating modes is greater than the number Np of fixed-voltage power sources.

47. The transmitter of claim 46 wherein the pulse train generator comprises a bipolar pulse train generator.

48. A computer-readable medium for generating a combined ultrasound image with a first imaging mode and a second imaging mode, the computer-readable medium comprising a data storage device and computer-executable program code stored on the data storage device, where the computer-executable program code comprises:
(a) code for generating transmit pulses at a predetermined voltage level for the first imaging mode;
(b) code for acquiring a first image in the first imaging mode in response to the transmit pulses generated by the code in (a);
(c) code for generating transmit pulses at the predetermined voltage level for the second imaging mode, with a duty cycle selected in response to one or more of: a restriction on surface temperature of a transducer, and a restriction on transducer power output;
(d) code for acquiring a second image in the second imaging mode in response to the transmit pulses generated by the code in (c); and
(e) code for displaying the first and second images.

49. The computer-readable medium of claim 48, where the code for generating transmit pulses at the predetermined voltage level for the first imaging mode comprises code for an on-off switching of a single DC voltage supply.

50. The computer-readable medium of claim 48, where the first imaging mode is a B-mode ultrasound imaging mode.

51. The computer-readable medium of claim 48, where the second imaging mode is a Doppler-spectral ultrasound imaging mode.

52. The computer-readable medium of claim 48, where the second imaging mode is a color-mode ultrasound imaging mode.

53. The computer-readable medium of claim 48, further comprising code for repeating the code in (a)-(e) to generate an interleaved sequence of first and second images.

54. The computer-readable medium of claim 48, where the duty cycle is selected to meet a maximum transducer surface temperature limit of 43° C.

55. The computer-readable medium of claim 48, where the duty cycle is selected to meet a maximum transducer power output of 720 mW/cm².

56. The computer-readable medium of claim 48, where the code in (a) includes code for transmitting a train of bipolar pulses.

57. The computer-readable medium of claim 48, where the code in (a) comprises
(a1) code for setting a voltage supply to the predetermined voltage level; and
(a2) code for using one or more switches to engage or disengage the voltage supply.

58. The computer-readable medium of claim 48, where the predetermined voltage level is a user-selectable voltage level.

59. The computer-readable medium of claim 48, where the predetermined voltage level is selected in response to one or more of: the restriction on surface temperature of the transducer, and the restriction on transducer power output.
